(19) United States
(12) Patent Application Publication
(10) Pub. No.: US 2026/0130616 A1
(43) Pub. Date: May 14, 2026

(54) DEVICE, METHOD AND NON-TRANSITORY COMPUTER-READABLE RECORDING MEDIUM FOR MONITORING BIOSIGNAL

(71) Applicant: HUINNO, CO., LTD., Seoul (KR)

(72) Inventors: Yeong Joon GIL, Busan (KR); Sung Hoon JUNG, Seoul (KR)

(21) Appl. No.: 19/383,603

(22) Filed: Nov. 8, 2025

Related U.S. Application Data
(63) Continuation of application No. PCT/KR2024/005616, filed on Apr. 25, 2024.

(30) Foreign Application Priority Data
May 10, 2023 (KR) 10-2023-0060263

(51) Int. Cl. A61B 5/282; A61B 5/00

(52) U.S. Cl. CPC A61B 5/282; A61B 5/684

(57) ABSTRACT

A device for monitoring a biosignal includes a main body unit; and an electrode unit being detachably coupled to the main body unit and including two or more electrodes for acquiring a biosignal. The main body unit is configured to determine an inter-electrode distance applied to a subject with reference to information on physical characteristics of the subject, and decide whether a distance between the two or more electrodes included in the electrode unit corresponds to the determined inter-electrode distance.

### CROSS-REFERENCE TO RELATED APPLICATIONS

This application is a Continuation of International Application No. PCT/KR2024/005616 filed on Apr. 25, 2024, which claims priority from Korean Patent Application No. 10-2023-0060263 filed on May 10, 2023. The aforementioned applications are incorporated herein by reference in their entireties.

### TECHNICAL FIELD

The present invention relates to a device, method, and non-transitory computer-readable recording medium for monitoring a biosignal.

### RELATED ART

Due to recent rapid progress in science and technology, the quality of life of all mankind is being enhanced and medical environment has changed a great deal. Particularly, in recent years, wearable monitoring devices that can monitor electrocardiogram (ECG) signals during daily life without visiting a hospital have become widely available to the public.

Conventional wearable monitoring devices employ predetermined off-the-shelf Ag/AgCl disposable electrodes, or are integrally configured such that an electrode unit is merged into a main body including circuitry for ECG signal monitoring. Therefore, most wearable ECG monitoring devices can only collect ECG signals from electrodes arranged in the same pattern, and cannot situationally adjust positions of the electrodes or distances between them.

According to previously reported research, the location or size of a subject's heart may vary depending on physical characteristics of the subject, resulting in significant differences (e.g., amplitude) in ECG signals measured from the subject. These differences hinder accurate analysis of the ECG signals.

In this connection, the inventor(s) have developed a technique capable of reducing deviations in biosignals due to a subject's physical characteristics by supporting adjustment of an inter-electrode distance of a wearable device to suit the subject's physical characteristics.

### SUMMARY

One object of the present invention is to solve all the above-described problems in the prior art.

Another object of the invention is to provide a device for monitoring a biosignal, the device comprising: a main body unit; and an electrode unit being detachably coupled to the main body unit and including two or more electrodes for acquiring a biosignal, wherein the main body unit is configured to determine an inter-electrode distance applied to a subject with reference to information on physical characteristics of the subject, and decide whether a distance between the two or more electrodes included in the electrode unit corresponds to the inter-electrode distance determined to be applied to the subject.

Yet another object of the invention is to reduce deviations in measured signals due to physical characteristics of a subject wearing a wearable monitoring device by supporting implementation of an inter-electrode distance suitable for the physical characteristics of the subject.

Still another object of the invention is to provide a wearable monitoring device capable of coping with physical characteristics of various subjects with a single main body unit by allowing electrode units having various inter-electrode distances to be detachably coupled to the main body unit.

The representative configurations of the invention to achieve the above objects are described below.

According to one aspect of the invention, there is provided a device for monitoring a biosignal, the device comprising: a main body unit; and an electrode unit being detachably coupled to the main body unit and including two or more electrodes for acquiring a biosignal, wherein the main body unit is configured to determine an inter-electrode distance applied to a subject with reference to information on physical characteristics of the subject, and decide whether a distance between the two or more electrodes included in the electrode unit corresponds to the determined inter-electrode distance.

According to another aspect of the invention, there is provided a method for monitoring a biosignal, the method comprising the steps of: determining an inter-electrode distance applied to a subject with reference to information on physical characteristics of the subject; and in response to a main body unit being detachably coupled to an electrode unit including two or more electrodes for acquiring a biosignal, deciding whether a distance between the two or more electrodes included in the electrode unit corresponds to the determined inter-electrode distance.

In addition, there are further provided other devices and methods to implement the invention, as well as non-transitory computer-readable recording media having stored thereon computer programs for executing the methods.

According to the invention, it is possible to drastically reduce deviations in measured signals due to physical characteristics of a subject wearing a wearable monitoring device by implementing an inter-electrode distance suitable for the physical characteristics of the subject.

According to the invention, it is possible to provide a wearable monitoring device capable of coping with physical characteristics of various subjects with a single main body unit by allowing electrode units having various inter-electrode distances to be detachably coupled to the main body unit.

### BRIEF DESCRIPTION OF THE DRAWINGS

FIG. 1 schematically shows the configuration of an entire system for monitoring a biosignal according to one embodiment of the invention.

FIG. 2 illustratively shows the configuration of a wearable device according to one embodiment of the invention.

FIG. 3 illustratively shows the configuration of a wearable device according to one embodiment of the invention.

FIG. 4 illustratively shows the configuration of a wearable device according to one embodiment of the invention.

FIG. 5 illustratively shows the internal configuration of a main body unit according to one embodiment of the invention.

### DESCRIPTION OF THE REFERENCE NUMERALS

100: communication network
200: biosignal analysis system
300: wearable device
310: main body unit
311: acquisition unit
312: decision unit
313: provision unit
314: communication unit
315: control unit
320: electrode unit
321: electrodes
322: circuit unit
323: pad
324: coupling unit
325: extension connection unit

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures, and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the invention will be described in detail with reference to the accompanying drawings to enable those skilled in the art to easily implement the invention.

### Configuration of the Entire System

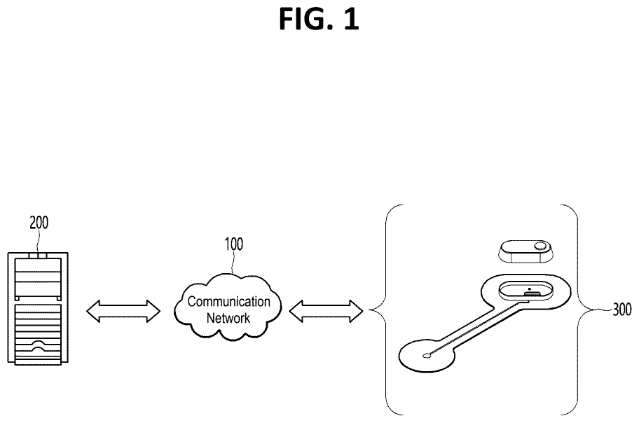

FIG. 1 schematically shows the configuration of the entire system for monitoring a biosignal according to one embodiment of the invention.

As shown in FIG. 1, the entire system according to one embodiment of the invention may comprise a communication network 100, a biosignal analysis system 200, and a wearable device 300.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, 5G communication, Bluetooth communication (including Bluetooth Low Energy (BLE) communication), infrared communication, and ultrasonic communication. As another example, the communication network 100 may be an optical communication network, at least a part of which may be implemented with a conventional communication scheme such as LiFi (Light Fidelity).

Next, the biosignal analysis system 200 according to one embodiment of the invention may communicate with the wearable device 300 to be described below via the communication network 100. Further, the biosignal analysis system 200 according to one embodiment of the invention may function to analyze health status of a subject (e.g., cardiac events such as arrhythmia) by receiving and analyzing a biosignal (e.g., an electrocardiogram signal) measured from the wearable device 300. Meanwhile, the biosignal analysis system 200 may be digital equipment having a memory means and a microprocessor for computing capabilities, and may be, for example, a server system operating on the communication network 100.

Next, the wearable device 300 according to one embodiment of the invention is digital equipment capable of connecting to and then communicating with the biosignal analysis system 200, and having a memory means and a microprocessor for computing capabilities, such as a smart patch, a smart watch, a smart band, and smart glasses, and may be a wearable monitoring device including a sensing means (e.g., a contact electrode) for measuring a biosignal (e.g., an electrocardiogram signal) from a human body.

In addition, according to one embodiment of the invention, the wearable device 300 may further include an application program for performing the functions according to the invention. The application may reside in the wearable device 300 in the form of a program module. The nature of the program module may be generally similar to those of program modules included in the biosignal analysis system 200 to be described below. Here, at least a part of the application may be replaced with a hardware or firmware device that may perform substantially equal or equivalent functions, as necessary.

### Configuration of the Wearable Device

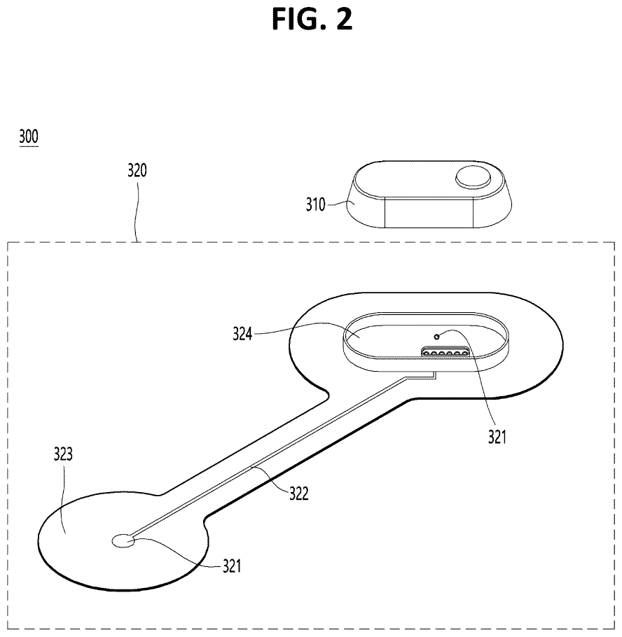

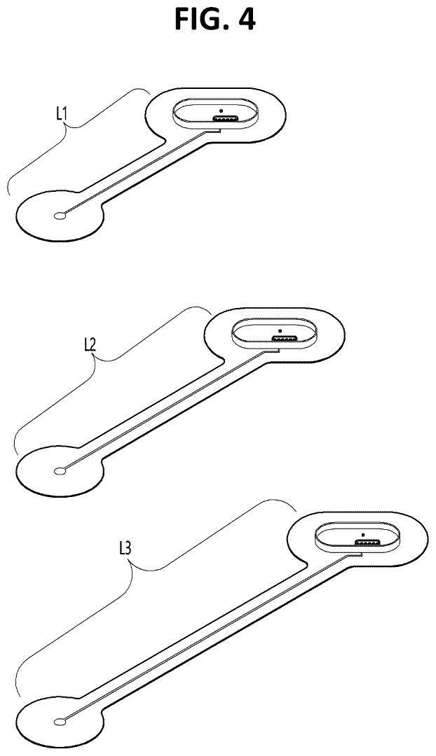

FIGS. 2 to 4 illustratively show the configuration of the wearable device according to one embodiment of the invention.

Referring to FIGS. 2 to 4, the wearable device 300 according to one embodiment of the invention may comprise a main body unit 310 and an electrode unit 320. Here, the electrode unit 320 according to one embodiment of the invention may include two or more electrodes 321, a circuit unit 322, and a pad 323. In the following, the electrode unit 320 will be discussed first and then the main body unit 310 will be discussed.

First, the electrode unit 320 according to one embodiment of the invention may include two or more electrodes 321 for acquiring a biosignal. Here, according to one embodiment of the invention, at least two electrodes 321 may be formed at a predetermined interval, and three electrodes including a reference electrode may preferably be formed. According to one embodiment of the invention, the electrodes 321 may form contact points with a patient's body to acquire a biosignal such as an electrocardiogram (ECG) signal, and the biosignal acquired from the electrodes 321 may be transmitted to the main body unit 310 through the circuit unit 322. For example, one of the two or more electrodes according to one embodiment of the invention may be formed at the bottom of a coupling unit 324 to acquire a biosignal.

Further, the electrode unit 320 according to one embodiment of the invention may include a circuit unit 322 for electrically connecting the electrodes 321 and the main body unit 310. Here, referring to FIG. 1, the circuit unit 322 according to one embodiment of the invention may include a circuit trace for transmitting the biosignal acquired from the electrodes 321 to the main body unit 310.

Furthermore, the electrode unit 320 according to one embodiment of the invention may include a pad 323. The pad 323 according to one embodiment of the invention may be arranged around the electrodes 321, and the electrodes 321 may form contact points with the patient's body when the pad 323 is attached to the patient, so that the electrodes 321 may acquire a biosignal. Here, the pad 323 according to one embodiment of the invention may include an attachment pad that is electrically connected to the circuit unit 322 and attached at a position appropriate for acquiring the patient's biosignal, and double-sided tape for attaching to the patient. For example, the biosignal according to one embodiment of the invention may be acquired by the electrodes 321 from the patient's body that is in contact with the attachment pad. Meanwhile, the attachment pad according to one embodiment of the invention may function to assist the electrodes 321 in acquiring the patient's biosignal by forming a contact point with the patient's body instead of the electrodes 321.

In addition, the coupling unit 324 according to one embodiment of the invention may function to allow the main body unit 310 and the electrode unit 320 to be detachably coupled. When the main body unit 310 and the electrode unit 320 are coupled by the coupling unit 324 according to one embodiment of the invention, the biosignal acquired from the electrode unit 320 may be transmitted to the main body unit 310, so that the main body unit 310 may store the biosignal.

Moreover, the biosignal according to one embodiment of the invention may include an electrocardiogram (ECG) signal. However, the biosignal according to the invention is not limited to an ECG signal, and may include various biosignals (e.g., blood oxygen saturation, heart rate, or body temperature) as long as the objects of the invention may be achieved.

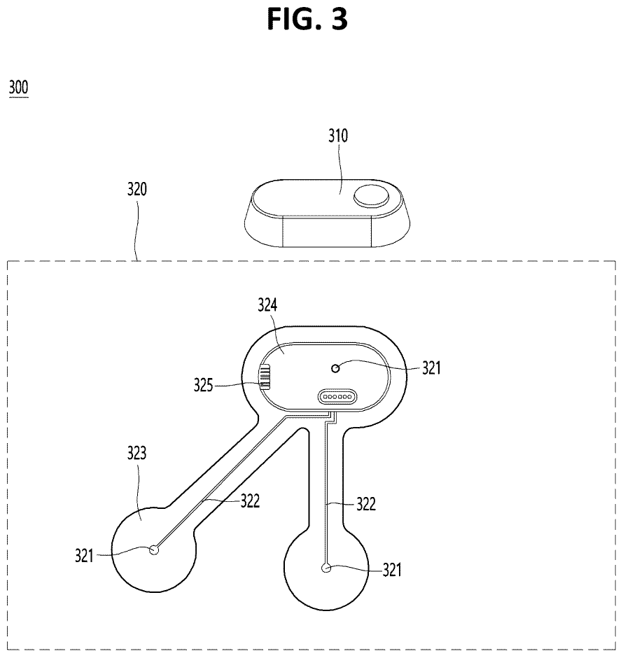

As shown in FIG. 3, the electrode unit 320 according to one embodiment of the invention may include three electrodes. Here, referring to FIG. 3, one electrode may be arranged at the bottom of the coupling unit 324, and the other two electrodes may be arranged at appropriate positions for acquiring an ECG signal. Here, a channel according to one embodiment of the invention may be defined by one electrode (i.e., one unipolar electrode) using a Wilson central terminal (WCT) or two electrodes (i.e., a pair of electrodes), and when the electrode unit 320 includes three electrodes (e.g., electrodes arranged at positions associated with RA (Right Atrium), LA (Left Atrium), and LL (Left Leg) of 12 ECG leads in the chest area), biosignals related to two channels (i.e., an RA-LA channel and an RA-LL channel) may be acquired. Further, the channel according to one embodiment of the invention may be defined as a lead specified by two electrodes.

Meanwhile, it should be understood that one of the electrodes 321 according to the invention does not necessarily have to be arranged at the bottom of the coupling unit 324, but may be arranged at various points for achieving the objects of the invention.

An extension connection unit 325 may be further formed in the electrode unit 320 according to one embodiment of the invention. Specifically, an extension connection unit 325 electrically connected to the electrode unit 320 may be further formed in the electrode unit 320, and additional electrodes for acquiring the biosignal may be connected to the extension connection unit 325.

As will be described later, according to one embodiment of the invention, an inter-electrode distance of the plurality of electrodes included in the electrode unit 320 may be variously set in two or more types. For example, the electrode unit 320 according to one embodiment of the invention may be any one of a first type (L1) electrode unit with a minimum inter-electrode distance, a second type (L2) electrode unit with an intermediate inter-electrode distance, and a third type (L3) electrode unit with a maximum inter-electrode distance. However, the type classification based on the inter-electrode distance of the electrode unit 320 according to the invention is not necessarily limited to the foregoing but may be changed without limitation as long as the objects of the invention may be achieved. As another example, the electrode unit 320 according to one embodiment of the invention may be classified into two types or four or more types according to the inter-electrode distance.

Next, the main body unit 310 according to one embodiment of the invention may include various components for operating the wearable device 300, such as a battery, a printed circuit board assembly (PCBA), a memory for storing the biosignal, and a processor for processing or analyzing the biosignal.

The main body unit 310 according to one embodiment of the invention may function to acquire unique information of the electrode unit 320 in response to being coupled to the electrode unit 320. Here, the unique information according to one embodiment of the invention may include information on the number or arrangement of the electrodes. Further, the unique information according to one embodiment of the invention may include information on the inter-electrode distance of the plurality of electrodes included in the electrode unit 320.

Meanwhile, the biosignal according to one embodiment of the invention is stored in an encrypted form, so that personal health information may be stored safely.

Finally, when a USB terminal is coupled to the main body unit 310 according to one embodiment of the invention and the main body unit is electrically connected to an external device (e.g., PC), the biosignal stored in the main body unit 310 may be transmitted to the PC. In this case, the transmitted biosignal according to one embodiment of the invention may be encrypted to safely protect personal health information.

Meanwhile, it should be understood that the term “patient” as used herein encompasses not only a patient with heart disease but also a patient who wants to check whether he/she has heart disease.

Hereinafter, the internal configuration of the main body unit 310 of the wearable device 300 crucial for implementing the invention and the functions of the respective components thereof will be discussed.

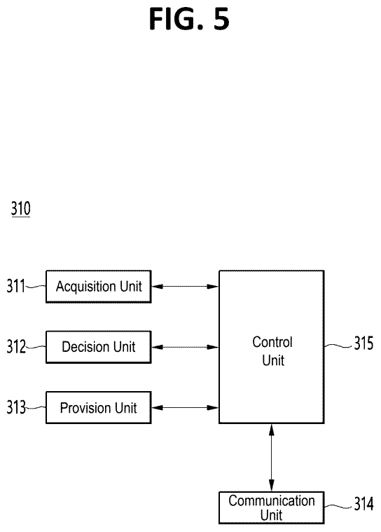

FIG. 5 illustratively shows the internal configuration of the main body unit according to one embodiment of the invention.

As shown in FIG. 5, the main body unit 310 according to one embodiment of the invention may comprise an acquisition unit 311, a decision unit 312, a provision unit 313, a communication unit 314, and a control unit 315. According to one embodiment of the invention, at least some of the acquisition unit 311, the decision unit 312, the provision unit 313, the communication unit 314, and the control unit 315 of the main body unit 310 may be program modules to communicate with an external system (not shown). The program modules may be included in the wearable device 300 in the form of operating systems, application program modules, and other program modules, while they may be physically stored in a variety of commonly known storage devices. Further, the program modules may also be stored in a remote storage device that may communicate with the wearable device 300. Meanwhile, such program modules may include, but are not limited to, routines, subroutines, programs, objects, components, data structures, and the like for performing specific tasks or executing specific abstract data types as will be described below in accordance with the invention.

Meanwhile, the above description is illustrative although the main body unit 310 has been described as above, and it will be apparent to those skilled in the art that at least a part of the components or functions of the main body unit 310 may be implemented in the biosignal analysis system 200 or a server (not shown) or included in an external system (not shown), as necessary.

First, the acquisition unit 311 according to one embodiment of the invention may acquire information on physical characteristics of a subject wearing the wearable device 300.

According to one embodiment of the invention, the information on the physical characteristics of the subject may include information on at least one of the subject's height, weight, and body mass index (BMI).

Specifically, according to one embodiment of the invention, the information on the physical characteristics of the subject may be inputted through a separate device (such as a smartphone held by the subject or an administrator) capable of communicating with the wearable device 300. When the subject or administrator inputs the information on the physical characteristics of the subject through the separate device, the information may be transmitted to the main body unit 310 of the wearable device 300 worn by the subject.

Further, the acquisition unit 311 according to one embodiment of the invention may acquire information on a distance between two or more electrodes included in the electrode unit 320 coupled to the main body unit 310.

Specifically, according to one embodiment of the invention, the information on the distance between the two or more electrodes included in the electrode unit 320 may be specified on the basis of unique information of the electrode unit 320, and the unique information of the electrode unit 320 may be acquired in response to the main body unit 310 being coupled to the electrode unit 320.

Next, the decision unit 312 according to one embodiment of the invention may determine an inter-electrode distance applied to the subject with reference to the information on the physical characteristics of the subject. Here, according to one embodiment of the invention, the information on the physical characteristics of the subject may include information on at least one of the subject's height, weight, and body mass index (BMI).

Specifically, when the inter-electrode distance is classified into three types (i.e., a minimum distance, an intermediate distance, and a maximum distance), the decision unit 312 according to one embodiment of the invention may determine the inter-electrode distance applied to the subject as one of the three types with reference to the subject's height, weight, or body mass index. For example, the three types of the inter-electrode distance, i.e., minimum distance (L1), intermediate distance (L2), and maximum distance (L3), may be set to 6 cm, 10 cm, and 12 cm, respectively.

More specifically, the decision unit 312 according to one embodiment of the invention may determine the inter-electrode distance applied to the subject as the minimum distance (L1) if the subject's height is less than a minimum threshold, and may determine the inter-electrode distance applied to the subject as the maximum distance (L3) if the subject's height is not less than a maximum threshold. For example, the minimum and maximum thresholds for the height may be 160 cm and 185 cm, respectively.

Further, the decision unit 312 according to one embodiment of the invention may determine the inter-electrode distance applied to the subject as the minimum distance (L1) if the subject's weight is less than a minimum threshold, and may determine the inter-electrode distance applied to the subject as the maximum distance (L3) if the subject's weight is not less than a maximum threshold. For example, the minimum and maximum thresholds for the weight may be 50 kg and 90 kg, respectively.

In addition, the decision unit 312 according to one embodiment of the invention may determine the inter-electrode distance applied to the subject as the intermediate distance (L2) if the subject's body mass index is less than a predetermined threshold, and may determine the inter-electrode distance applied to the subject as the maximum distance (L3) if the subject's body mass index is not less than the predetermined threshold. For example, the predetermined threshold for the body mass index may be 25.

Meanwhile, the decision unit 312 according to one embodiment of the invention may determine the inter-electrode distance applied to the subject in consideration of priority among two or more physical characteristics of the subject. For example, the decision unit 312 according to one embodiment of the invention may treat the height as the most important determinant among the height, weight, and body mass index, and treat the body mass index as the least important determinant.

Although the thresholds for the height, weight, and body mass index considered in determining the type of inter-electrode distance applied to the subject have been described above by way of illustration, it is noted that the configuration according to the invention is not necessarily limited to the foregoing but may be changed without limitation as long as the objects of the invention may be achieved.

Further, the decision unit 312 according to one embodiment of the invention may decide whether the distance between the two or more electrodes included in the electrode unit 320 coupled to the main body unit 310 corresponds to the inter-electrode distance determined to be applied to the subject.

Next, the provision unit 313 according to one embodiment of the invention may generate feedback information on whether the distance between the two or more electrodes included in the electrode unit 320 corresponds to the inter-electrode distance for the subject, according to a result of the decision of the decision unit 312. Specifically, if the distance between the two or more electrodes included in the electrode unit 320 coupled to the main body unit 310 does not correspond to the inter-electrode distance applied to the subject, the provision unit 313 according to one embodiment of the invention may generate feedback information to be provided to the subject or administrator to inform that the electrode unit 320 with an inter-electrode distance unsuitable for the subject is coupled to the main body unit 310. For example, if the subject's height is less than the minimum threshold so that the electrode unit 320 with the minimum inter-electrode distance is suitable for the subject, but the electrode unit 320 with the maximum inter-electrode distance is coupled to the main body unit 310, the provision unit 313 according to one embodiment of the invention may generate feedback information to the effect that the inter-electrode distance of the electrode unit 320 is not suitable for the subject. Accordingly, the subject or administrator will be able to ensure that the electrode unit 320 with an inter-electrode distance suitable for the subject is coupled to the main body unit 310 on the basis of the feedback information.

Therefore, the subject may wear the wearable device 300 provided with the electrode unit 320 having an inter-electrode distance suitable for physical condition of the subject.

Next, the communication unit 314 according to one embodiment of the invention may function to enable data transmission/reception from/to the acquisition unit 311, the decision unit 312, and the provision unit 313.

Lastly, the control unit 315 according to one embodiment of the invention may function to control data flow among the acquisition unit 311, the decision unit 312, the provision unit 313, and the communication unit 314. That is, the control unit 315 according to one embodiment of the invention may control data flow into/out of the main body unit 310 or data flow among the respective components of the main body unit 310, such that the acquisition unit 311, the decision unit 312, the provision unit 313, and the communication unit 314 may carry out their particular functions, respectively.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks, and magnetic tapes; optical media such as compact disk-read only memory (CD-ROM) and digital versatile disks (DVDs); magneto-optical media such as floptical disks; and hardware devices such as read-only memory (ROM), random access memory (RAM), and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A device for monitoring a biosignal, the device comprising:
a main body unit; and
an electrode unit being detachably coupled to the main body unit and including two or more electrodes for acquiring a biosignal,
wherein the main body unit is configured to determine an inter-electrode distance applied to a subject with reference to information on physical characteristics of the subject, and decide whether a distance between the two or more electrodes included in the electrode unit corresponds to the determined inter-electrode distance.

2. The device of claim 1, wherein the biosignal includes an electrocardiogram (ECG) signal.

3. The device of claim 1, wherein the information on the physical characteristics of the subject may include information on at least one of the subject's height, weight, and body mass index (BMI).

4. The device of claim 1, wherein information on the distance between the two or more electrodes included in the electrode unit is specified on the basis of unique information of the electrode unit.

5. The device of claim 4, wherein the unique information is acquired in response to the main body unit being coupled to the electrode unit.

6. The device of claim 1, wherein the main body unit is configured to generate feedback information on whether the distance between the two or more electrodes included in the electrode unit corresponds to the determined inter-electrode distance, according to a result of the decision.

7. A method for monitoring a biosignal, the method comprising the steps of:
determining an inter-electrode distance applied to a subject with reference to information on physical characteristics of the subject; and
in response to a main body unit being detachably coupled to an electrode unit including two or more electrodes for acquiring a biosignal, deciding whether a distance between the two or more electrodes included in the electrode unit corresponds to the determined inter-electrode distance.

8. The method of claim 7, wherein the biosignal includes an electrocardiogram (ECG) signal.

9. The method of claim 7, wherein the information on the physical characteristics of the subject may include information on at least one of the subject's height, weight, and body mass index (BMI).

10. The method of claim 7, wherein information on the distance between the two or more electrodes included in the electrode unit is specified on the basis of unique information of the electrode unit.

11. The method of claim 10, wherein the unique information is acquired in response to the main body unit being coupled to the electrode unit.

12. The method of claim 7, further comprising the step of generating feedback information on whether the distance between the two or more electrodes included in the electrode unit corresponds to the determined inter-electrode distance, according to a result of the decision.

13. A non-transitory computer-readable recording medium having stored thereon a computer program for executing the method of claim 7.
